SEQUENCE LISTING (18 sequences)

SEQ ID NO: 1 - DNA, 4181 bp, Artificial Sequence
  Description of Artificial Sequence Synthetic construct pCMV-PL-N1
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggtcgccaccatgagctccaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaaccggactcagatctcgagctcaagcttcgaattctgcagtcgacggtaccgcgggcccgggatccaccggatctagataactgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaagatcgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgccatgcat

SEQ ID NO: 2 - DNA, 156 bp, Artificial Sequence
  Description of Artificial Sequence Synthetic construct ProLabel oligonucleotide
tcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaa

SEQ ID NO: 3 - protein, 587 aa, Artificial Sequence
  Description of Artificial Sequence GLUT4- ProLabel protein
MPSGFQQIGSEDGEPPQQRVTGTLVLAVFSAVLGSLQFGYNIGVINAPQKVIEQSYNETWLGRQGPEALVPRGSSLAVVLQRRDWENPGVTQLNRLAAHPPFASWRNSEEARTDRPSQQLRSLNGEKLVPRGSGILYPYDVPDYAGPSSIPPGTLTTLWALSVAIFSVGGMISSFLIGIISQWLGRKRAMLVNNVLAVLGGSLMGLANAAASYEMLILGRFLIGAYSGLTSGLVPMYVGEIAPTHLRGALGTLNQLAIVIGILIAQVLGLESLLGTASLWPLLLGLTVLPALLQLVLLPFCPESPRYLYIIQNLEGPARKSLKRLTGWADVSGVLAELKDEKRKLERERPLSLLQLLGSRTHRQPLIIAVVLQLSQQLSGINAVFYYSTSIFETAGVGQPAYATIGAGVVNTVFTLVSVLLVERAGRRTLHLLGLAGMCGCAILMTVALLLLERVPAMSYVSIVAIFGFVAFFEIGPGPIPWFIVAELFSQGPRPAAMAVAGFSNWTSNFIIGMGFQYVAEAMGPYVFLLFAVLLLGFFIFTFLRVPETRGRTFDQISAAFHRTPSLLEQEVKPSTELEYLGPDEND

SEQ ID NO: 4 - DNA, 10 bp, Artificial Sequence
  Description of Artificial Sequence Kozak sequence
rccaccatgg

SEQ ID NO: 5 - protein, 5 aa, Artificial Sequence
  Description of Artificial Sequence Formula peptide
XXGXG

SEQ ID NO: 6 - protein, 9 aa, Artificial Sequence
  Description of Artificial Sequence HA epitope tag
YPYDVPDYA

SEQ ID NO: 7 - protein, 4 aa, Artificial Sequence
  Description of Artificial Sequence Illustrative peptide
DEVD

SEQ ID NO: 8 - protein, 4 aa, Artificial Sequence
  Description of Artificial Sequence Illustrative peptide
DDVD

SEQ ID NO: 9 - protein, 4 aa, Artificial Sequence
  Description of Artificial Sequence Illustrative peptide
VEMX

SEQ ID NO: 10 - protein, 4 aa, Artificial Sequence
  Description of Artificial Sequence Illustrative peptide
EVQX

SEQ ID NO: 11 - protein, 4 aa, Artificial Sequence
  Description of Artificial Sequence Illustrative peptide
WEHD

SEQ ID NO: 12 - protein, 6 aa, Artificial Sequence
  Description of Artificial Sequence Illustrative peptide
KKRKRR

SEQ ID NO: 13 - protein, 10 aa, Artificial Sequence
  Description of Artificial Sequence Illustrative peptide
GSGIFLETSL

SEQ ID NO: 14 - protein, 6 aa, Artificial Sequence
  Description of Artificial Sequence Illustrative peptide
LVPRGS

SEQ ID NO: 15 - protein, 4 aa, Artificial Sequence
  Description of Artificial Sequence Illustrative peptide
IEGR

SEQ ID NO: 16 - protein, 5 aa, Artificial Sequence
  Description of Artificial Sequence Illustrative peptide
DDDDK

SEQ ID NO: 17 - protein, 7 aa, Artificial Sequence
  Description of Artificial Sequence Illustrative peptide
LEVLFXP

SEQ ID NO: 18 - protein, 5 aa, Artificial Sequence
  Description of Artificial Sequence Formula peptide
XXGGX

(19) United States
(12) Patent Application Publication
(10) Pub. No.: US 2005/0136488 A1
(43) Pub. Date: Jun. 23, 2005

(54) Cellular membrane protein assay

(76) Inventors: Joseph L. Horecka, Fremont, CA (US); Richard M. Eglen, Los Altos, CA (US)

Correspondence Address: PETERS VERNY JONES & SCHMITT, L.L.P., 425 SHERMAN AVENUE, SUITE 230, PALO ALTO, CA, 94306, US

(21) Appl. No.: 10/981,320

(22) Filed: Nov. 3, 2004

Related U.S. Application Data
(60) Provisional application No. 60/517,663, filed on Nov. 6, 2003.

(51) Int. Cl. G01N 33/53; G01N 33/567

(52) U.S. Cl. 435/7.2

(57) ABSTRACT

Methods and compositions are provided for determining cell membrane protein populations in the cell membrane of a cell and changes in the population due to changes in the environment or status of the cell. The methods employ a cell having a fusion construct of the cell membrane protein linked to a signal producing peptide through an exofacial protease recognition site or sites. The signal producing peptide is either an enzyme fragment capable of binding to a second enzyme fragment to form an active enzyme when released from the cell membrane or has two binding sites, where the complementary binding entities are related in that a signal is produced when the two entities are in proximity. For the enzyme signal producing peptide, by adding the protease to the cell and the second enzyme fragment and substrate, one can determine the cell membrane protein population and the effect of changes in the cell environment on such population. Similarly, by adding the two entities and any other necessary reagents, a signal is produced whereby one can determine the cell membrane protein population and the effect of changes in the cell environment on such population.

This application claims priority of Provisional patent application Ser. No. 60/517,663, filed Nov. 6, 2003.

### BACKGROUND OF THE INVENTION

1. Field of the Invention

The invention relates generally to methods of measuring surface membrane protein populations.

2. Background Information

Cells communicate with their environment, among other methods, through proteins in the cellular membrane that extend into the extracellular environment. These membrane proteins often have cavities or surfaces with specific binding affinities, 10⁶ M⁻¹, for ligands. Binding of the ligand to the membrane protein, usually referred to as a receptor, results in a change in conformation of the receptor that results in the transduction of a signal into the cytoplasm. The signal may be a result of binding to a protein, activation resulting in enzymatic activity, release of a protein complexed with the receptor, and the like. The ligand and the receptor then become separated, usually by endocytosis of the complex of the receptor and ligand. Frequently, the ligand is a protein that is degraded in a lysosome and the freed receptor is then returned to the cellular membrane.

The population of cell membrane proteins is affected by numerous changes in the environment of the cell and the physiology of a cell. Events associated with the role of the cell and the response to the presence of a drug, infectious agent, or in the event of stimulation or deactivation, frequently lead to an increase or decrease in the cell surface membrane protein population. Thus, down or up regulation, degradation, transport to different compartments, etc., can all lead to changes in the protein population at the surface. Such population can serve as a monitor of various events occurring in the cell and affecting cellular activity.

Many of the therapeutic attempts involve binding of compounds to a receptor in place of the natural ligand, where the compound may play the part of an agonist or antagonist. The effect of the ligand may be to transduce a signal, fill the binding site to prevent binding of the ligand or cause endocytosis resulting in a reduction in the population of receptors at the surface.

Therapeutic efforts are frequently directed to diminishing or enhancing the population or availability of a cellular receptor. CD34 with its binding to HIV is only one of many cellular membrane proteins that plays a detrimental role in an infectious disease. There is, therefore, substantial interest in being able to determine the population of proteins on a cell surface and the effect of a change in environment or cell status on such population.

Methods of determining the population of proteins, particularly receptors, at the membrane surface should be adaptable to single determinations, as well as being capable of being used in high throughput screening. Today, drug companies need to screen large numbers of compounds for their activity, as well as whether the compounds have undesirable side effects. Therefore, the number of determinations before screening a compound in vivo has grown astronomically. By using robotics and sophisticated software, large numbers of assays can be performed and the results tabulated to provide structure/activity information.

Because of the large numbers of determinations to be performed, the cost of reagents becomes a factor in the employment of a particular protocol. Methods that are likely to be employed will be sensitive to small variations in the population of the membrane protein and should allow for amplification of the signal for each molecule at the surface. In addition, the assay should be robust, desirably use materials with which laboratories are familiar and comfortable and have an easy protocol that can be readily automated with a minimum number of steps requiring handling.

There is, therefore, an interest in developing assays that fulfill many of the objectives for use as single tests as well as high throughput screening.

Relevant Literature

There are numerous references concerned with the use of β fragments in assay systems. The following are illustrative. Douglas, et al., Proc. Natl. Acad. Sci. USA 1984, 81:3983-7 describes the fusion protein of ATP-2 and lacZ. WO92/03559 describes a fusion protein employing α-complementation of β-galactosidase for measuring proteinases. WO01/0214 describes protein folding and/or solubility assessed by structural complementation using the α-peptide of β-galactosidase as a fusion protein. WO01/60840 describes fusion proteins including a fusion protein comprising an enzyme donor β-galactosidase for measuring protein folding and solubility. Homma, et al., Biochem. Biophys. Res. Commun., 1995, 215, 452-8 describes the effect of α-fragments of β-galactosidase on the stability of fusion proteins. Abbas-Terki, et al., Eur. J. Biochem. 1999, 266, 517-23 describes α-complemented β-galactosidase as an in vivo model substrate for the molecular chaperone heat-shock protein in yeast. Miller, et al., Gene, 1984, 29, 247-50 describe a quantitative β-galactosidase α-complementation assay for fusion proteins containing human insulin β-chain peptides. Thomas and Kunkel, Proc. Natl. Acad. Sci. USA, 1993, 90, 7744-8 describe an ED containing plasmid to measure mutation rate.

### SUMMARY OF THE INVENTION

Methods and compositions are provided that allow for the determination of populations of proteins, usually receptors, at cellular membranes. The methods comprise the use of a transformed viable cell having genetic capability to express a fusion protein comprising a cellular membrane protein fused to a signal producing polypeptide through a proteolytic susceptible sequence. The signal producing peptide is usually detected after being released from the surface membrane through the specific proteolytic susceptible sequence and a proteinase that cleaves the specific sequence, where the presence of the cell surface substantially inhibits the production of a signal. The expression construct may use the naturally occurring transcriptional regulatory region or a different region depending upon the purpose of the determination. After changing the environment of the cell, one can determine the population of the membrane protein by measuring the signal producing polypeptide. One may also determine the amount of cellular membrane protein that has been endocytosed by lysing the cell. Of particular interest is using as the signal producing polypeptide an enzyme fragment that is inhibited from complexing with a second fragment to form the active enzyme by the cellular membrane, e.g. a fragment of β-galactosidase.

### BRIEF DESCRIPTION OF THE DRAWINGS

FIG. 1A provides the nucleic acid sequence of DiscoveRx cloning vector pCMV-PL-N1 (SEQ ID: NO. 1);

FIG. 1B provides the nucleic acid sequence encoding ProLabel (SEQ ID: NO. 2);

FIG. 1C is a plasmid map of pGLUT4-PL.1 with features and restriction enzyme sites relevant to the cloning;

FIG. 2 is a translation of the GLUT4-ProLabel gene fusion in plasmid pGLUT4-PL.1 (SEQ ID: NO. 3);

FIG. 3A is a Western blot detection of GLUT4-PL in CHO cells. FIG. 3B is an identically prepared blot was probed with anti-actin antibody to control for equivalent sample loading;

FIG. 4 is a graph showing that thrombin treatment of intact CHO/pGLUT4-PL.1 cells leads to a dose-dependent increase in EFC activity (▪). Inactivation of thrombin by AEBSF completely blocks the effect (Δ);

FIG. 5 is a graph showing that active thrombin protease is compatible with EA and EFC. In the first step of the assay, intact cells were treated with buffer alone or buffer containing increasing amounts of thrombin. In the second step, half the samples were treated with EA alone (●) and half were treated with EA containing AEBSF to inactivate thrombin (▾);

FIG. 6 is a graph showing that thrombin treatment does not affect cell integrity in the intact-cell assay. Cells expressing GLUT4-PL (▪) were assayed in parallel with control cells expressing IkB-PL (▾), a cytoplasmic reporter protein. Lysis of cells expressing IkB-PL shows a marked increase in EFC activity (data not shown);

FIG. 7 is a bar graph showing thrombin cleavage releases ProLabel in a soluble form from intact cells expressing GLUT4-PL. FIG. 7A shows intact-cell reaction products separated into supernatant and cell fractions (FIG. 7B), the latter prepared as a detergent lysate, and then assayed for EFC activity;

FIG. 8 is a bar graph showing insulin-dependent translocation of GLUT4-PL to the cell surface. CHO/pGLUT4-PL.1 cells were treated for 30 min with serum-containing media alone or the same with insulin at the indicated concentrations. Cells were subsequently processed with the intact-cell EFC assay using thrombin (FIG. 8A). A parallel set of samples was assayed without thrombin (FIG. 8B); the thrombin-independent signal is also insulin-independent; and

FIG. 9A is a bar graph with background-subtracted data revealing an increased insulin response. FIG. 9B shows thrombin- and insulin-independent signal (averaged from the data in the lower graph of FIG. 8) subtracted as background from the data derived from thrombin-treated samples. Numbers above the bars represent the percent increase of signal relative to the no-insulin control.

### DETAILED DESCRIPTION OF THE INVENTION

Methods for determining protein populations at cellular membranes are provided using viable cells having the genetic capability to express a cellular membrane fusion protein. The method will be homogeneous in the sense of not requiring a separation step from components associated with the production of the signal. The cells that are employed are genetically modified to be able to express the fusion protein that comprises a cellular membrane protein fused to a signal producing peptide linked through at least one protease consensus sequence for release from the cell surface. The signal producing peptide cleaved from the cell surface is measured. Normally, the proximity of the signal producing peptide to the cell membrane surface substantially reduces the ability of the signal producing peptide to produce a signal by binding to its complementary member. A substantial increase in signal is observed when the signal producing peptide is released from the cellular membrane protein and the released signal producing peptide can successfully bind to its complementary member. Therefore the released signal producing peptide can be measured in the presence of the signal producing peptide that remains bound to the surface, as a measure of the amount of fusion protein present on the cell membrane surface.

The expression construct will provide for a transcriptional regulatory sequence, and a fusion construct, referred to below as the protein reagent. The fusion construct will normally be under the transcriptional regulatory control of the transcriptional regulatory sequence. The transcriptional regulatory sequence will have a promoter, usually including a TATA box and a CAAT box, may frequently include an enhancer and may be constitutive or inducible. The regulatory region may be an endogenous regulatory region, particularly the native regulatory region where one is interested in the effect of an environment change on the transcription, or an exogenous regulatory region, particularly when one is interested in the effect of an environment change on endocytosis and/or restoration to the membrane after endocytosis and/or up- or down regulation of expression and/or transport to a compartment of the cell. There are numerous commercially available regulatory regions, including strong and weak regulatory regions, regulatory regions associated with housekeeping proteins, viral proteins, mutated regulatory regions, e.g. temperature sensitive regulatory regions, etc.

Sequences 5′ to the start codon may include sequences associated with enhanced expression. Such sequences include a Kozak sequence, 5′-A/GCCACCATGG-3′ (SEQ ID NO: 4), where the underlined nucleotides define the start codon.

The insertion construct can take many forms depending upon whether it is inserted into the membrane protein encoding sequence or fused at or proximal to the 5′ or 3′ terminus of the membrane protein encoding sequence and/or is joined by a linker sequence. The primary elements of the insertion construct include coding, e.g., a leader sequence, to provide for transport of the expression product to the cell membrane, a signal producing sequence to provide the signal for detection of the presence of the cell membrane protein at the cell membrane, a protease consensus sequence for cleavage by a protease to release the signal producing sequence from the cell membrane protein, and the cell membrane protein or surrogate fragment thereof. Other encoding capabilities may be included such as linker sequences, epitope sequences, additional protease recognition sequences, etc.

The insertion sequence of the fusion construct will normally have coding for transport to the cell surface membrane. Commonly, this is a nucleic acid 5′-sequence encoding a leader sequence for transport of the fusion protein to the cell membrane. A wide variety of leader sequences are available and the leader sequence selected may be the leader sequence of the cell membrane protein or a different endogenous or exogenous protein, usually endogenous protein. Leader sequences usually comprise terminal polar, usually anionic amino acids, joined by a lipophilic chain, where the leader sequence will about 20±2 amino acids. In addition, there will be at least one transmembrane sequence and there may be a plurality of transmembrane sequences, where the protein may have one or more exofacial loops.

Optionally, following the leader sequence will be a linker sequence. The linker sequence may have from 1 to 90 codons, usually not more than about 70 codons. The polypeptide linker sequence may serve a number of functions, aiding in the assembly of the fusion protein encoding nucleic acid, providing stability when cleaved with the enzyme donor sequence that follows in the 5′-3′ direction, providing an epitope to further identify the fusion construct, and the like. The linker sequence may be a portion of the first exofacial sequence of the cell membrane protein, where the linker sequence may be inserted without interfering with the response from the change in environment or may be in an exofacial loop, where it is joined directly or through linking amino acids to two transmembrane sequences. In addition, the linker sequence may include a protease consensus sequence, so as to remove all or a portion of the linker sequence from the signal producing peptide. At the 5′-end of the linker sequence and protease consensus sequence, the consensus sequence may abut the signal producing peptide or be not more than about 40, usually not more than about 30 amino acids from the signal producing peptide.

Between the signal producing peptide and the cell membrane protein residue, there is a protease consensus sequence, so that in the presence of the protease the signal producing sequence is freed from the cell membrane protein and released into the medium. Since the cell membrane is a large sterically inhibiting entity, release from the cell membrane surface will greatly facilitate the complexing between the signal producing peptide and the complementary member of the signal producing system.

The essential element of the insertion construct is the signal producing peptide and the protease consensus sequences(s) linking the signal producing peptide to the cell membrane protein. As indicated above, other capabilities may be built into the insertion sequence. The insertion sequence may be the N terminal of the cell membrane protein or inserted into an exofacial domain of the N- or C-terminal domain extending into the medium or any loop of the surface membrane protein.

In a preferred aspect, the signal producing peptide will be referred to as the enzyme donor. The signal producing peptide is one of a pair of fragments of an enzyme that is reconstituted when the two fragments, the enzyme donor (“ED”) and the enzyme acceptor (“EA”) complex together. The ED will be a fragment of an enzyme that can be complemented with another fragment, the EA, to form an active enzyme. There are two different situations. In a first situation, the ED and EA complex to form the active enzyme in the absence of any ancillary binding. The ED and EA individually are substantially inactive, but when combined independently complex to form the active enzyme. In the other situation, the fragments of the enzyme are fused to auxiliary polypeptides that independently complex, and when the auxiliary polypeptides complex, the enzyme fragments complex to form an active enzyme. As in the first situation, the enzyme fragments are substantially inactive individually, but as distinguished from the first case, when the two enzyme fragments are brought together in the absence of the auxiliary polypeptides, the fragments do not complex to form an active enzyme.

The indicator enzymes formed by the ED and EA and their ED and EA fragments are required to have a number of characteristics. First, the fragments should be substantially inactive, in that there should be little, if any, background with only one fragment present in the presence of substrate. Second, the fragments have sufficient affinity for each other, so that upon scission of the protein reagent. the released ED will combine with EA to provide an active enzyme. The ED fragment of the protein reagent will complex with the EA fragment as a result of the affinity of the fragments of the enzyme for each other or as a result of being fused to auxiliary binding entities that will bring the enzyme fragments together resulting in an active enzyme. That is, in the former case, the enzyme fragments are capable of complexing without having an auxiliary binding entity to bring the fragments together to form a complex. In the latter case, the enzyme fragments will not independently form a complex, but when the auxiliary proteins form a complex, the enzyme fragments are then able to form an active enzyme.

Various indicator enzymes are known that fulfill these criteria and additional enzymes may be developed in accordance with known technologies. Indicator enzymes that fit these criteria include β-galactosidase (See, U.S. Pat. No. 4,708,929), ribonuclease A (See, U.S. Pat. No. 4,378,428), where the smaller fragment may come from the amino or carboxy terminus or internally, β-lactamase WO 00/71702 and 01/94617 and Wehrman, et al., Proc. Natl. Acad. Sci. 2002, 99, 3469-74, or enzymes that have small peptide cofactors, such as adenovirus proteases (See, U.S. Pat. No. 5,935,840). To identify other indicator enzymes that can serve in place of the above indicator enzymes, enzyme genes may be cleaved asymmetrically to define a small and large fragment and expressed in the same and different cells. In the presence of the substrate, the cells producing both fragments would catalyze the reaction of the substrate, while there should be little, if any turnover, with the individual fragments. Alternatively, one may express the fragments individually and if there is no reaction, combine the mixtures to see whether an enzyme-catalyzed reaction occurs.

Indicator enzymes of interest are those having subunits that are below about 300 kDa, generally below about 150 kDa. The independently complexing small fragment will be under 15 kDal, more usually under about 10 kDal, frequently under about 125 amino acids, generally under about 100 amino acids and preferably not more than about 75 amino acids. Depending on the enzyme the independently complexing ED may be as small as 10 amino acids, usually being at least about 25, more usually at least about 35 amino acids. With this criterion in mind, the fragments that are screened can be selected to provide the appropriately sized small fragment.

The enzymes having fragments that complex in conjunction with a fused auxiliary protein will generally have fragments having from 20-80%, more usually 25-75% of the amino acids of the enzyme. The fragments may be modified by the addition of from about 1 to 20, usually 2 to 10, amino acids to enhance the affinity of the fragments during complexation. Enzymes that provide for low affinity complexation to an active enzyme include β-galactosidase, β-glucuronidase, Staphylococcal nuclease, and β-lactamase, as exemplary. The binding proteins may have as few as 8, more usually at least 10 amino acids and may be 150, usually not more than about 100 kDal. Binding proteins may include homo- and heterodimers, epitopes and immunoglobulins or fragments thereof, e.g. Fab, ligands and receptors, etc. In some instances, complexation may require the addition of an additional reagent, so that complexation with formation of an active enzyme does not occur to any significant degree in the absence of the additional reagent, e.g. FK1012, cyclosporin and rapamycin.

Each of the indicator enzymes will have an appropriate substrate. α-galactosidase uses β-galactosylethers having as the aglycone, a masked fluorescer or chemiluminescent agent that become unmasked upon hydrolysis of the glycosidic ether. Ribonuclease A, fluorescer modified nucleotides, exemplified by uridine 3′-(4-methylumbelliferon-7-yl)ammonium phosphate, adenovirus proteinase, -(L, I, M)-X-G-G/X- or -(L, I, M)-X-G-X/G- (SEQ ID NO: 5), where the vertical line denotes the position of cleavage; the P3 (X) position appears to be unimportant for cleavage (Anderson, C. W., Virology, 177; 259 (1990); Webster, et al., J. Gen. Virol., 70; 3225 (1989)) and the peptide substrate can be designed to provide a detectable signal, e.g. using fluorescence resonance energy transfer, by having a fluorescer and a quencher on opposite sides of the cleavage site. β-glucuronidase substrates are exemplified by 5-Br-4-Cl-3-indolyl β-D-glucuronidase.

Since β-galactosidase is paradigmatic of the peptides used in the subject invention, demonstrating the criteria for having two peptides that when combined complex non-covalently to form an active enzyme, this enzyme will be frequently referred to hereafter as illustrative of the class, except for those situations where the different enzymes must be considered independently. The ED for β-galactosidase is extensively described in the patent literature. U.S. Pat. Nos. 4,378,428; 4,708,929; 5,037,735; 5,106,950; 5,362,625; 5,464,747; 5,604,091; 5,643,734; and PCT application nos. WO96/19732; and WO98/06648 describe assays using complementation of enzyme fragments. The β-galactosidase ED will generally be of at least about 35 amino acids, usually at least about 37 amino acids, frequently at least about 40 amino acids, and usually not exceed 100 amino acids, more usually not exceed 75 amino acids. The upper limit is defined by the effect of the size of the ED on the performance and purpose of the determination, the activity of the fragment and the complex, and the like.

Instead of having ED as the signal producing peptide, one may have oligopeptides having two binding sites, where a signal is produced when both of the binding sites are occupied. Occupation of the two binding sites is inhibited by the presence of the cell membrane surface to the signal producing peptide, so that upon release from the cell surface membrane, a substantial increase in signal is observed. The two binding sites can be any convenient peptide site, such as a biotin mimic, polyhistidine, histidine/cysteine complexing combinations, ligands, epitopes, or other relatively small, less than about 5 kDal oligopeptides that have complementary binding partner that will generally be greater than about 5 kDal, usually greater than about 10 kDal. The two binding sites will be separated by a linker so that their individual binding to their complementary binding partners will not be inhibited, but interactions between the binding partners will be permitted. Therefore, the binding sites will usually be separated by at least about 5 amino acids, usually at least about 10 amino acids and not more than about 50 amino acids, usually not more than about 30 amino acids.

Complementary binding members may be binding pairs, such as biotin and streptavidin, chelating oligopeptides and nickel derivatives, ligands and receptors, epitopes and immunoglobulins and fragments thereof, e.g. Fab, Fv, etc. Each of these have found extensive exemplification in the literature to form complexes for a variety of reasons, both associated with and unassociated with diagnostic determinations. See, for example, U.S. Pat. Nos. 5,260,203 and 6,312,699 and Gissel, et al., 1995 J Pept Sci 1, 212-26; Suigara, et al., 1998 FEBS Lett 426, 140-4; and Honey, et al., 2001 Nucl. Acids. Res 29, E24.

There are a large number of assays that depend for their producing a signal on having two different entities in propinquity. These include a light absorbing and energy transferring entity and an energy receiving and light emitting or fluorescent entity (referred to as “FRET”); two enzymes where the product of one is the substrate of the other and the final product is fluorescent or chemiluminescent; transfer of a metastable species that reacts to produce a detectable signal, etc. See, for example, U.S. Pat. Nos. 4,663,278; 4,822,733; 5,811,311; 5,830,769; and 6,406,913.

The signal producing entities, such as the fluorescers, enzymes, etc., may be bound to particles, such as latex particles, gold sol, carbon, etc., where the increased bulk will further hinder the binding of the signal producing entity to the cell membrane surface. In this way, lower backgrounds can be achieved. There will be the consideration that both of the entities must bind to the released signal producing peptide, but this can be readily achieved by using a single particle or by the appropriate spacing between the binding entities of the signal producing peptide.

The cell membrane protein may be any protein of interest where the population of the cell membrane protein is of scientific or therapeutic interest. Thus proteins of interest include receptors, channels, transporters, adhesion proteins, proteins involved with cell-cell interactions, proteins involved with binding of infectious agents, MHC proteins, proteins associated with diapedesis, etc. The protein may be bound to the membrane through a transmembrane sequence or through a lipid, e.g. myrisotyl, fatty acid substituted glycerol, farnesyl, etc., or other mechanism for holding the protein in proximity to the membrane. These sequences encoding for post-translational processing are well known and are described in numerous texts and articles. See, for example, Reuther, et al., 2000 Meth Enzymol 327, 331-50; van't Hoff and Rich, 2000 ibid 327, 317-330; and Hofemeister, et al. 2000 Mol Cell Biol 11, 3233-46.

The cell membrane proteins or their truncated or modified analogs may have a single contact with the cell membrane, such as a transmembrane sequence or a lipid anchoring the protein to the cell membrane surface. With some cell membrane proteins, the protein extends through the membrane multiple times, so that there will be multiple coding sequences for the transmembrane sequences. Depending upon what one is determining, one may be interested in having the entire cell membrane protein, only the N-terminal portion of the protein, the wild-type protein or mutated protein.

Specific proteins or groups of proteins of interest include glucose transporters, GPCR proteins, adhesion proteins, and hormone binding proteins, e.g., insulin receptor.

The protease enzymes that are employed can be selected somewhat arbitrarily. The protease enzymes should be fairly selective in their cleavage site, that is have a relatively infrequent sequence as their consensus sequence, preferably should not cleave the cell membrane protein rather than the recognition sequence, should have a high turnover rate, not be inhibited by the presence of the cell membrane, and be robust and readily available. Also, it may or may not be an enzyme secreted by the cell, so that endogenous enzymes may find employment.

Enzymes of interest include serine/threonine hydrolases, cysteine hydrolases, metalloproteinases, BACEs (e.g., α-, β- and γ-secretases). Included within these classes are such protein groups as caspases, the individual MMPs, elastases, collagenases, ACEs, carboxypeptidases, blood clotting related enzymes, complement components, cathepsins, dipeptidyl peptidases, granzymes, etc. For other enzyme groups, see Handbook of Proteolytic Enzymes, ed. A J Barnet, N D Rowland, and J F Woessner. Other types of enzymes include abzymes.

Specific serine proteases include neutrophil elastase, involved in pulmonary emphysema, leukocyte elastase, tyrosine carboxypeptidase, lysosomal carboxypeptidase C, thrombin, plasmin, dipeptidyl peptidase IV; metalloproteinases include carboxypeptidases A and B, angiotensin converting enzyme, involved in hypertension, stromelysin, involved with inflammatory disorders, e.g. rheumatoid arthritis, P. aeruginosa elastase, involved in lung infections; aspartic proteases include renin, involved in hypertension, cathepsin D, HIV protease; cysteine proteases include lysosomal carboxypeptidase, cathepsin B, involved in cell proliferative disorders, cathepsin G, cathepsin L, calpain, involved with brain cell destruction during stroke; etc.

The proteases may come from any convenient source and may be involved with various processes, such as infections and replication of the infectious agent, viral, bacterial, fungal, and protista; phagocytosis, fibrinolysis, blood clotting cascases, complement cascades, caspase cascades, activation of proforms of proteins, protein degradation, e.g. ubiquitinated proteins, apoptosis, etc., cell growth, attachment, synaptic processes, etc. The proteases may come from a variety of sources, prokaryotes, eukaryotes or viruses, depending on the nature of the assay.

As already indicated, the organisms from which the proteases are naturally derived are varied. Among viruses, the proteases may be derived from HIV-1, and -2, adenoviruses, hepatitis viruses, A, B, C, D and E, rhinoviruses, herpes viruses, e.g. cytomegalovirus, picornaviruses, etc. Among unicellular microorganisms are Listeria, Clostridium, Escherichia, Micrococcus, Chlamydia, Giardia, Streptococcus, Pseudomonas, etc. Of course, there are numerous mammalian proteases of interest, particularly human proteases.

There are numerous scientific articles describing proteases and their substrates. Illustrative articles are as follows, whose relevant content is specifically incorporated herein by reference. Among the metalloproteinases are MMP-2, having target sequences L/IXXXHy; XHySXL; and HXXXHy (where Hy intends a hydrophobic residue), Chen, et al., J. Biol. Chem., 2001. Other enzymes include mitochondrial processing peptidase, having the target sequence RXXAr (where Ar is an aromatic amino acid), Taylor, et al., Structure 2001, 9, 615-25; caspases, VAD, DEVD and DXXD, as well as the RB protein, Fattman, et al., Oncogene 2001, 20, 2918-26, DDVD of HPK-1, Chen, et al., Oncogene 1999, 18, 7370-7; VEMD/A and EVQD/G of Keratins 15 and 17, Badock, et al., Cell Death Differ. 2001, 8, 308-15; WEHD of pro-interleukin-1β, Rano, et al., Chem. Biol. 1997, 4, 149-55; furin, KKRKRR of RSV fusion protein, Zimmer, et al., J. Biol. Chem. 2001, 20, 2918-26; HIV-1 protease, GSGIF*LETSL, Beck, et al., Virology 2000, 274, 391-401. Other enzymes include thrombin, LVPRGS, Factor Xa protease, IEGR, enterokinase, DDDDK, 3C human rhinovirus protease, LEVLFQ/GP.

Other references describing proteases include: Rabay, G. ed., “Proteinases and their Inhibitors in Cells and Tissues, 1989, Gustav Fischer Verlag, Stuttgart; Powers, et al., in “Proteases—Structures, Mechanism and Inhibitors,” 1993, Birkhauser Verlag, Basel, pp. 3-17; Patick and Potts, Clin. Microbiol. Rev. 1998, 11, 614-27; Dery, et al., Am. J. Physiol. 1998, 274, C1429-52; Kyozuka, et al., Cell Calcium 1998, 23, 123-30; Howells, et al., Br. J. Haematol. 1998, 101, 1-9; Hill and Phylip, Adv. Exp. Med. Biol. 1998, 436, 441-4; Kidd, Ann. Rev. Physiol. 1998, 60, 533-73; Matsushita, et al., Curr. Opin. Immunol. 1998, 10, 29-35; Pallen and Wren, Mol. Microbiol. 1997, 26, 209-21; DeClerk, et al., Adv. Exp. Med. Biol. 1998, 425, 89-97; Thomberry, Br. Med. Bull. 1997, 53, 478-90, which references are specifically incorporated herein.

Besides the naturally occurring recognition sequences, using combinatorial approaches, one can design recognition sequences that will have specificity for one or a family of enzymes. By preparing a library of oligopeptides that are labeled and having an array of the labeled oligopeptides where the location identifies the sequence, one need only add the protease of interest to the array and detect the release of the label. Having microwell plates, with the oligopeptides bound to the surface and labeled with a fluorescer, allows one to follow cleavage by internal reflection of activating irradiation. Numerous other approaches can also be used. By using synthetic sequences, one can optimize the cleavage for a particular protease. By using a plurality of protein reagents, one can obtain profiles that will be specific for specific enzymes.

Various cells may be employed for performing the assay. The cells may be from any source, but will mainly be mammalian, although other eukaryotes and prokaryotes may find use. The cells may be primary cells, cell lines, immortalized cells, or the like. The cells will be matched with the transcriptional regulatory region to allow for transcription and the construct may be modified to have codons preferred by the host cell. Illustrative cells sources include primate, e.g. human, chimpanzee, etc., rodent, mouse, rat and hamster, domestic animal, bovine, ovine, porcine, canine and feline, etc. The cell membrane protein may be endogenous or exogenous to the host. While for the most part, one will be interested in the expression of the endogenous protein, the subject methodology is applicable to any situation where a change in environment results in a change in the population of a cell membrane protein. For example, if one is solely interested in the effect of a change in environment on transcription factors, then the protein is not a significant factor in studying the effect of the change of environment on the transcription factor, rather the protein serves as a surrogate for determining the effect on the transcription factor. Alternatively, if one is interested in the effect of a ligand binding a receptor, then the protein receptor will normally be essential to the assay.

The expression construct may be illustrated by the following formula:
(a) LS-Lₐ-IS-(N)RCMP or (b) LS-Lₐ-IS-(C)RCMP, where N and C intend the N- or C-terminus respectively
where:
LS is codons encoding the leader sequence;
L is a linker of from 1 to 70 codons in reading frame with the leader sequence, where the linker may be a polypeptide unassociated with the cell membrane protein, a portion of the cell membrane protein, may include a protease consensus sequence, or may encode for some other function, e.g., an epitope;
a is 0 or 1, indicating the presence or absence of the linker;
IS is the insertion sequence and includes at least the signal producing sequence and the protease consensus sequence, namely SPS-RS, where SPS intends the signal producing sequence and RS intends the protease recognition or consensus sequence, with the RS bound to the RCMP; and
RCMP intends the residual portion of the cell membrane protein, which may include the entire protein where the IS binds directly to the N-terminus of the cell membrane protein or may be inserted into the first exofacial region of the cell membrane protein or into a loop of the cell membrane protein, where the linker would be a portion of the cell membrane protein. In some instances, rather than have the IS bound to the N-terminal portion of the cell membrane protein, it may be expeditious to have the IS bound to the C-terminal portion of the protein, where the C-terminus is exofacial. In that case the formula would be reversed as indicated for formula (b).

The insertion sequence will normally be at least about 45 codons or amino acids, usually at least about 50 codons or amino acids and not more than about 250 codons or amino acids, more usually not more than about 200 codons or amino acids. The RS will generally be at least about two codons or amino acids, usually at least about four codons or amino acids and not more than about 36, usually not more than about 20 codons or amino acids, although only one codon or amino acid is required with Endoproteinase Lys-C, where only a single lysine is required.

The expression construct is produced in accordance with conventional ways, as described in various laboratory manuals and by suppliers of vectors that are functional in numerous hosts. See, for example, Sambrook, Fritsch & Maniatis, “Molecular Cloning: A Laboratory Manual,” Second Edition (1989) Cold Spring Harbor Laboratory Press, Cold Spring Harbor, N.Y. (herein “Sambrook et al., 1989”); “DNA Cloning: A Practical Approach,” Volumes I and II (D. N. Glover ed. 1985); “Oligonucleotide Synthesis” (M. J. Gait ed. 1984); “Nucleic Acid Hybridization” [B. D. Hames & S. J. Higgins eds. (1985)]; “Transcription And Translation” [B. D. Hames & S. J. Higgins, eds. (1984)]; “Animal Cell Culture” [R. I. Freshney, ed. (1986)]; “Immobilized Cells And Enzymes” [IRL Press, (1986)]; B. Perbal, “A Practical Guide To Molecular Cloning” (1984).

Vectors that may be used include viruses, plasmids, cosmids, phagemids, YAC, BAC and HAC. Other components of the vector may include origins of replication for one or more hosts, expression constructs for selection, including antibiotic resistance, proteins providing for a signal, etc., integration sequences and enzymes providing for the integration, multiple cloning sites, expression regulatory sequences, expression construct for a protein of interest, particularly where the protein is coordinately or differentially expressed in relation to the protein reagent, sequences allowing for ready isolation of the vector, etc. Commercially available vectors have many or all of these capabilities and may be used to advantage.

The DNA or RNA vectors may be introduced into a cellular host, whereby the expression of the fusion protein can occur. The host may be a primary cell, a cell line, a unicellular microorganism, or the like, where the cell may be modified having an expression construct integrated or transiently present in the cell expressing a secretable form of EA, expressing or over-expressing a protein that the cell does not normally express under the conditions of the assay, not expressing a protein that the cell normally expresses as a result of a knockout, transcription or translation inhibitor, or the like.

The gene encoding the fusion protein will be part of an expression construct. The gene is positioned to be under transcriptional and translational regulatory regions functional in the cellular host. In many instances, the regulatory regions may be the native regulatory regions of the gene encoding the protein that forms the EC (expression construct), where the fusion protein may replace the native gene. The site of the gene in an extrachromosomal element or in the chromosome may vary as to transcription level. Therefore, in many instances, the transcriptional initiation region will be selected to be operative in the cellular host, but may be from a virus or other source that will not significantly compete with the native transcriptional regulatory regions or may be associated with a different gene from the gene for the EC, which gene will not interfere significantly with the transcription of the fusion protein.

It should be understood that the site of integration of the expression construct, if integrated into a host chromosome, would affect the efficiency of transcription and, therefore, expression of the fusion protein. One may optimize the efficiency of expression by selecting for cells having a high rate of transcription, one can modify the expression construct by having the expression construct joined to a gene that can be amplified and coamplifies the expression construct, e.g. DHFR in the presence of methotrexate, or one may use homologous recombination to ensure that the site of integration provides for efficient transcription. By inserting an insertion element into the genome, such as Cre-Lox at a site of efficient transcription, one can direct the expression construct to the same site. In any event, one will usually compare the enzyme activity from cells in a predetermined environment to cells in the environment being evaluated.

The vector will include the fusion gene under the transcriptional and translational control of a promoter, usually a promoter/enhancer region, optionally a replication initiation region to be replication competent, a marker for selection, and may include additional features, such as restriction sites, PCR initiation sites, an expression construct providing constitutive or inducible expression of EA, or the like. As described above, there are numerous vectors available providing for numerous different approaches for the expression of the fusion protein in a host.

The vector may be introduced into the host cells by any convenient and efficient means, such as transfection, electroporation, lipofection, fusion, transformation, calcium precipitated DNA, etc. The manner in which the vector is introduced into the host cells will be one of efficiency and convenience in light of the nature of the host cell and the vector and the literature has numerous directions for the introduction of a vector into a host cell and the selection of the host cells that have effectively received the vector. By employing expression constructs that allow for selection, e.g. antibiotics, the cells may be grown in a selective medium, where only the cells comprising the vector will survive.

The assay procedure employed is to use the intact cells, either viable or non-viable. Non-viability can be achieved by heat, antibiotics, toxins, etc., which induce mortality while leaving the cells intact. The cells are grown in culture in an appropriate culture medium suitable for the cells and may be grown to confluence or subconfluence, e.g. 80%. The fusion protein expression construct and other constructs, as appropriate, may be present in the cell, integrated into the genome or may be added transiently by the various methods for introducing DNA into a cell for functional translation. These methods are amply exemplified in the literature, as previously described. By employing a marker with the protein reagent for selection of cells comprising the construct, such as antibiotic resistance, development of a detectable signal, etc., cells in culture comprising the fusion protein can be separated from cells in which the construct is absent. Once the fusion protein is being expressed, the environment of the cells may be modified, as appropriate.

In carrying out the assay, candidate compounds may be added to a cell containing mixture, changes in the culture medium may be created, other cells may be added for secretion of factors or binding to the transformed cells, viruses may be added, or the like. After sufficient time for changes in the environment to take effect, the medium may optionally be aspirated off and the cells allowed to incubate with a protease to permit the protease to cleave the fusion protein. The cleaved fragment is then assayed with an assay cocktail comprising EA and enzyme substrate, and the signal from the product is read. One can then relate this signal with the signal produced in the absence of the candidate compound. Alternatively, reagents are added that bind to the cleaved fragment, so as to be brought into close proximity that allows for the determination of the amount of fragment released from the cell surface, e.g. a pair of fluorescers that provide fluorescence resonance energy, enzyme or metastable species channeling, etc.

During incubation with the protease other components associated with the activity of the protease may be present, e.g. buffers to provide the desired pH, and the sample mixture is incubated, conveniently at a controlled temperature, which may include room temperature, for at least 1 min, usually at least about 5 min and not more than about 90 min, usually not more than about 60 min, there being no advantage in unduly extending the incubation period. When the assay is performed in a 96-well plate, the number of cells present will generally be in the range of about 10³-10⁵ and the volume of the cell medium will generally be in the range of about 10 to 100 μl.

If not already present EA is added in a volume of about 5 to 50 μl and the mixture incubated for at least about 5 min, usually at least about 10 min and not more than about 60 min, usually not more than about 45 min. Generally the amount of EA will be at least equal to the highest concentration of the ED anticipated to be formed, usually in excess, generally about 10-fold excess or more, more usually not more than about 10-fold excess. If not already present about 5 to 50 μl of a substrate providing a detectable signal is then added, where the substrate is in substantial excess of the amount that will be turned over in the assay. Illustrative substrates, many of which are commercially available, include dyes and fluorescers, such as X-gal, CPRG, 4-methylumbelliferonyl β-galactoside, resorufin β-galactoside, Galacton Star (Tropix, Applied Biosystems). The procedure follows the conventional procedure for other analytes described in the scientific and patent literature. See, for example, U.S. Pat. Nos. 4,708,929 and 5,120,653, as illustrative. The assay mixture may then be read at a specific time, e.g. 1-10 min, or as a rate, taking readings at specific intervals. With a chemiluminescent readout, the signal may be integrated for a time period of from 0.1 s to 1 min.

For the alternative signal producing polypeptides, the appropriate reagents are added as is conventional in the field and as described in the cited references. For example, for the fluorescence resonance energy transfer, one could use a fluorescer bound to a particle that emits at a wavelength that another fluorescer absorbs, followed by emission. By employing a combination of an epitope and biotin mimic, one would use a particle with both an antibody and the absorbing entity and streptavidin with the fluorescing entity. For the enzyme channeling, one could have the first enzyme that produces the product which is the substrate for the second enzyme bound to streptavidin and the second enzyme bound to a particle to which an antibody is also bound. In the case of the metastable species, one can have an enzyme producing singlet oxygen and a compound that reacts with singlet oxygen to emit light.

For convenience, kits can be provided that include the genetic construct, particularly as a vector that provides transient expression of the construct, i.e. the fusion construct gene under the control of a transcriptional and translational regulatory region or cells comprising such construct, the protease for releasing the signal producing peptide, and the other reagents, such as the enzyme acceptor and substrate or the two reagents that interact with the signal producing peptide to provide a signal. Also directions in written or electronic form can be provided for performing the assay.

While much of the experimental work was done with the human glucose transporter, GLUT4, it is intended to be paradigmatic of the surface membrane proteins that can be measured and also illustrates the trafficking of surface membrane proteins, where the population of the surface membrane proteins can be up or down regulated. Similarly, endocytosis can change the population at the surface.

### Experimental

The following examples are intended to illustrate but not limit the invention.

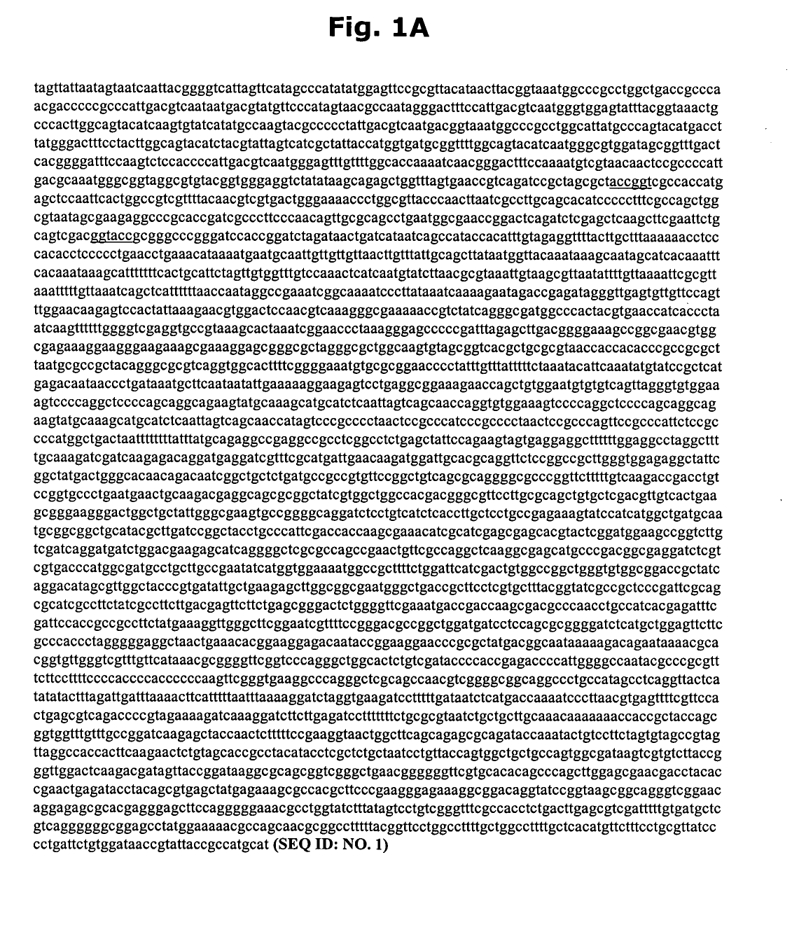

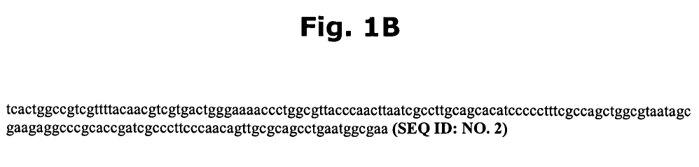

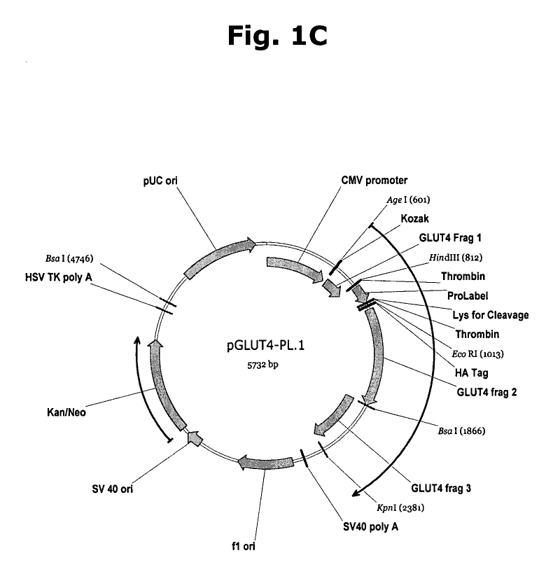

Cloning of the GLUT4-PL construct. A cloning strategy was designed to create a GLUT4-ProLabel fusion gene under the control of the CMV promoter in pCMV-PL-N1, a commercially available cloning vector (DiscoveRx, Fremont, Calif.) whose nucleic acid sequence is shown in FIG. 1A. Unique AgeI and KpnI restriction sites flanking the fusion gene were incorporated so that the ORF can be excised in toto and transferred to another expression vector, if desired. A Kozak consensus sequence was included immediately 5′ of the GLUT4 start codon to facilitate efficient translation. ProLabel® (ProLabel is the registered trademark for the enzyme donor fragment of E. coli β-galactosidase having the nucleic acid sequence shown in FIG. 1B), was inserted following GLUT4 codon 67, a position chosen because of successful reports in the literature of inserting single (HA and myc) and multiple tandem (7×myc) epitope tags at this site (Quon, et al., Proc. Natl. Acad. Sci. USA, 1994, 91, 5587-91; Bogan, et al., Mol Cell Biol., 2001, 21, 4785-806; Kanai, et al., J. Biol. Chem., 1993 5, 268, 14523-6). Thrombin cleavage sites flanking ProLabel allow for its proteolytic release from whole cells in which the GLUT4 fusion protein has been transported to the cell surface. A single lysine residue was inserted immediately following ProLabel that, together with a lysine residue naturally present at codon 50 in the first exofacial loop of GLUT4 provides a second means of proteolytic release of ProLabel using Endoproteinase Lys-C.

The Thrombin-ProLabel-Lys-Thrombin DNA (where thrombin indicates the cleavage consensus sequence) cassette is flanked by unique HindIII (upstream) and EcoRI (downstream) restriction sites, allowing for the simple swapping of it with virtually any cassette encoding ProLabel flanked by other protease cleavage sites. An HA epitope tag (YPYDVPDYA) (SEQ ID NO: 6) inserted following the cleavable ProLabel cassette allows for detection of the fusion protein by conventional immunological techniques. In total, 77 codons (encoding Thrombin-ProLabel-Lys-Thrombin-HA, and including codons associated synthetic cloning sites) were inserted between codons 67 and 68 in GLUT4. Finally, the 3′ region of the GLUT4 ORF was engineered to remove intron 7 sequences present in the commercial GLUT4 cDNA (NIH MGC clone IMAGE ID No. 5187454; obtained from Open Biosystems, Huntsville, Ala.).

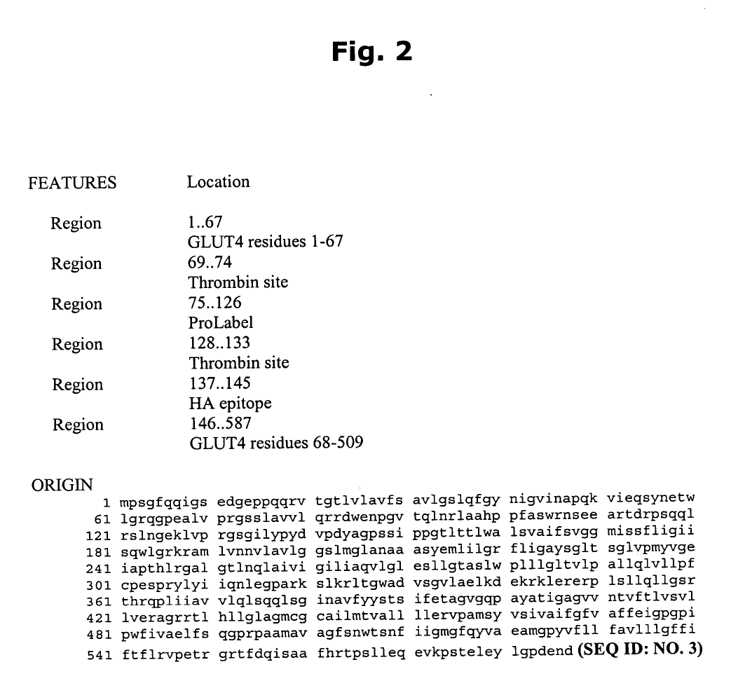

The plasmid described above was constructed using DNA fragments obtained by PCR amplification from GLUT4 cDNA and ProLabel templates with custom PCR primers. In total, four PCR-amplified fragments were created and cloned into DiscoveRx vector pCMV-PL-N1. Recombinant clones were analyzed by restriction enzyme mapping and DNA sequencing of the entire insert region. A correct clone was identified and saved as plasmid pGLUT4-PL.1. A plasmid map with features and restriction enzyme sites relevant to the cloning is shown in FIG. 1C. Translation of the GLUT4-ProLabel gene fusion with annotated features is shown in FIG. 2.

Expression of GLUT4-PL. Functional studies of pGLUT4-PL.1 were carried out in transiently transfected CHO cells. These studies included: 1) detection of the expressed protein on a Western blot, 2) development and characterization of an intact-cell EFC (enzyme fragment complementation) assay using thrombin protease, and 3) application of the assay to detect insulin-dependent translocation of GLUT4-PL to the cell surface.

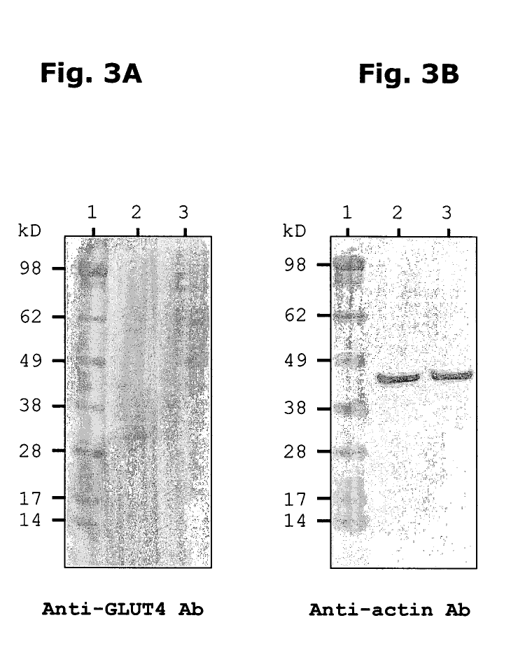

Western blot analysis was carried out to confirm expression of GLUT4-PL in CHO cells transiently transfected with pGLUT4-PL.1. The predicted molecular weight of the fusion protein is 63.5 kDal. Anti-GLUT4 polyclonal antibody detected polypeptides in a total cell lysate ranging in size from ˜33 kDal to just over 62 kDal (FIGS. 3A and 3B). Specificity of the antibody was demonstrated by the lack of staining of a lysate prepared from control cells expressing EGFP.

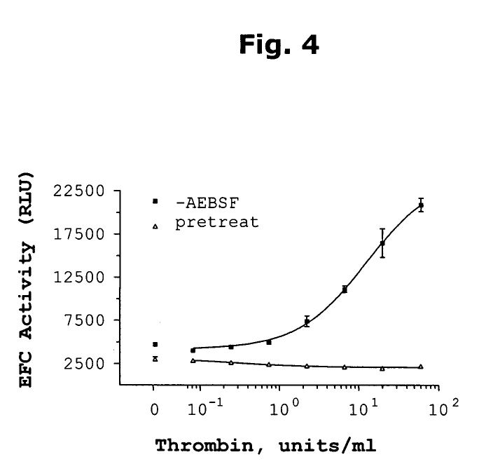

Detection of GLUT4-PL after protease cleavage. Central to the concept of using EFC to monitor GLUT4-PL at the cell surface is the proteolytic release of the internal ProLabel tag from the protein's first exofacial loop. Initial studies were therefore directed at developing and characterizing an intact-cell EFC protocol using thrombin protease. All of the experiments described below were carried out in 96-well assay plates. To test whether thrombin treatment could enhance EFC signal, intact cells expressing pGLUT4-PL.1 were treated with 50 μl buffer alone or buffer containing thrombin at increasing concentrations. Eighty μl of a solution containing EA and the protease inhibitor AEBSF (7.5 mM final concentration) were added subsequently, followed by 30 μl of chemiluminescent substrate. Thrombin treatment led to a dose-dependent enhancement of EFC activity, with 60 units/ml enhancing EFC activity 4.4-fold over untreated cells (FIG. 4). To test whether thrombin proteolytic activity per se, and not a non-specific component of the thrombin formulation was responsible for the increased EFC signal, a control experiment was performed by inactivating thrombin with AEBSF prior to its addition to cells. Inactivated thrombin had no signal enhancement activity (FIG. 4).

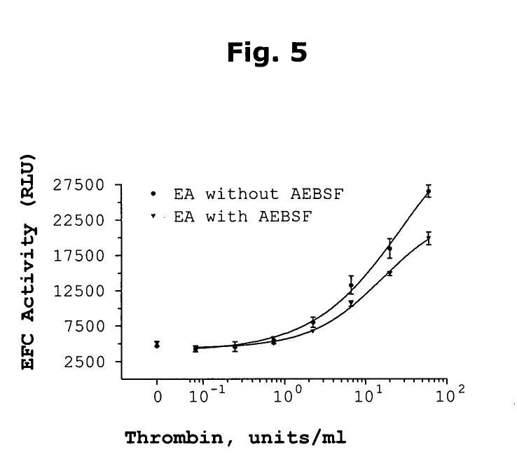

In the initial intact-cell EFC protocol, the protease inhibitor AEBSF was used to inactivate thrombin in the EA addition step because it was not known whether active thrombin would inhibit EA, for example, by non-specific cleavage of the EA polypeptide. An experiment comparing EA formulated with and without AEBSF tested the compatibility active EA and thrombin (FIG. 5). We found that EA formulated without AEBSF gave higher EFC activity, which probably reflects the continued proteolytic release of ProLabel during the EA incubation step. The finding that active thrombin and EA are compatible implies that the thrombin cleavage and EA addition steps can be combined.

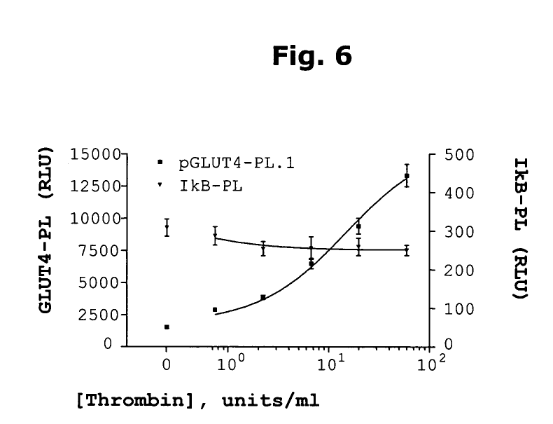

To demonstrate that thrombin does not affect cell integrity in the intact-cell protocol, we assayed HeLa cells expressing the cytoplasmic reporter protein IκB-PL; when lysed, these cells produce a high EFC signal. CHO/pGLUT4-PL.1 and HeLa/IκB-PL cells were assayed in parallel with a series of increasing thrombin concentrations (FIG. 6). As had been observed above, thrombin treatment of CHO/pGLUT4-PL.1 cells led to a dose-dependent increase in EFC activity. In contrast, no such increase was observed with the HeLa/IκB-PL cells, demonstrating that thrombin does not affect cell integrity.

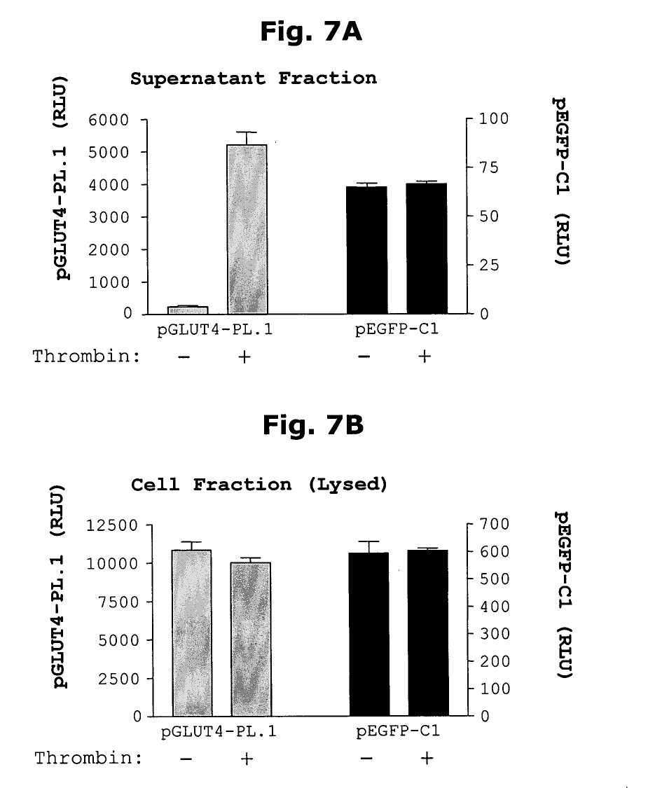

To biochemically demonstrate that thrombin cleavage releases ProLabel from the surface of intact cells, we separated the reaction products into two fractions: the liquid above the intact cells (supernatant) and the remaining cell fraction (tested as a detergent lysate). CHO/pGLUT4-PL. 1 cells were seeded into two 6 cm dishes. On the day of assay, the media was removed and the cells were washed once with PBS. To one dish was added buffer only, to the other buffer containing thrombin at 60 units/ml. After 1.5 hrs incubation at 37° C., the liquid above the cells was carefully collected, spiked with AEBSF to inactivate thrombin, and cleared of possible whole-cell contaminants by two sequential, low-speed centrifugations. The adherent cells in the dish were washed once for 15 min with PBS containing AEBSF and then lysed with a CHAPS-based lysis buffer containing AEBSF. As a control, a pair of plates seeded with CHO/pEGFP cells (no ProLabel) was processed in parallel to follow the endogenous β-galactosidase activity present in CHO cells. We found a significant increase in EFC activity in the supernatant fraction of CHO/pGLUT4-PL.1 cells that had been treated with thrombin (FIGS. 7A and 7B). This result demonstrates that ProLabel is released from the cell surface by thrombin and implies that both of the thrombin cleavage sites flanking ProLabel are recognized and cleaved. Examining the cell fraction as a detergent lysate, we found that the EFC activity remaining in the CHO/pGLUT4-PL.1 sample treated with thrombin was only slightly reduced relative to that of the untreated sample (FIGS. 7A and 7B); this slight reduction might reflect the partitioning of only a small fraction of GLUT4-PL to the cell surface under basal growth conditions.

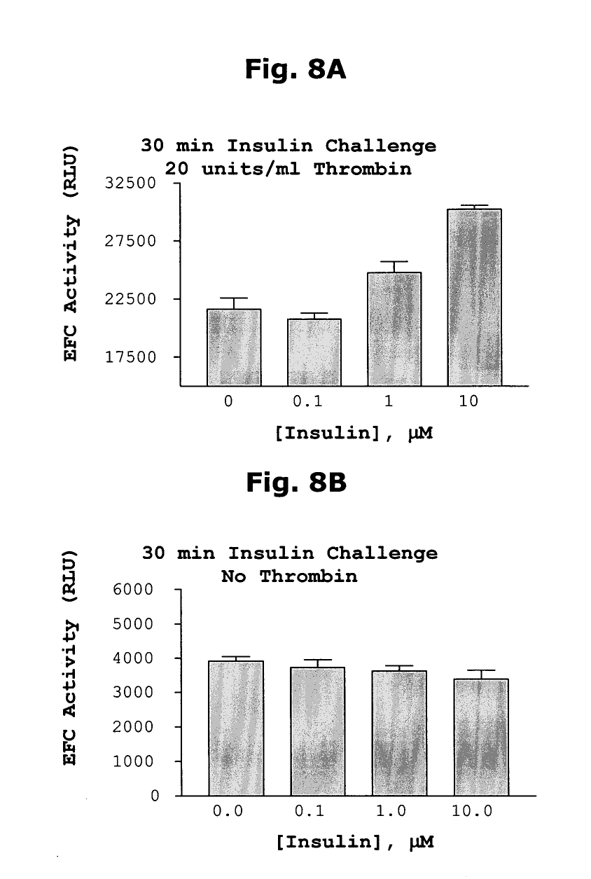

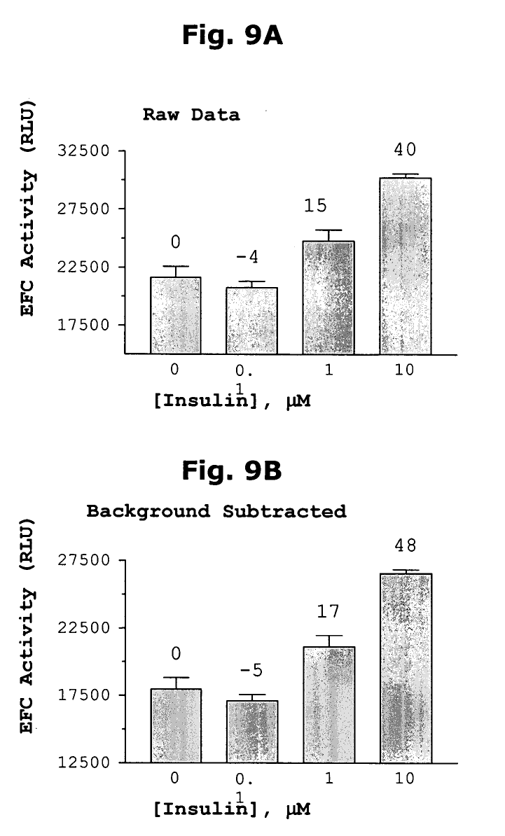

Effect of insulin on GLUT4-PL localization. The above experiments served to develop and characterize a protocol for detecting GLUT4-PL at the cell surface under basal growth conditions. We next tested whether exogenously added insulin would increase the fraction of GLUT4-PL present at the cell surface. Insulin is known to stimulate the transport of GLUT4 from intracellular compartments to the cell surface (for review, see Bryant, et al., Nature Reviews 2002, 3, 267-77). In this experiment, CHO/pGLUT4-PL.1 cells were treated for 30 min with 0, 0.1, 1, and 10 μM insulin in serum-containing media. The liquid above the cells was then replaced with buffer containing thrombin at 20 units/ml and processed for the intact-cell EFC assay. The two sets of samples treated with 1 and 10 μM insulin showed a 15% and 40% increase, respectively, in EFC activity relative to the no-insulin control. An insulin-dependent increase was only observed in the thrombin-treated samples: a parallel set of samples processed without thrombin showed no such increase (FIGS. 8A and 8B). Subtracting the thrombin- and insulin-independent signal as background reveals an increased insulin response (FIGS. 9A and 9B).

Paradigmatic protocols were established for CHO cells and 96-well assay plates as follows:

1) Seed cells into individual wells at a density of 10,000 cells in 100 μl media. For transient transfectants, replace the media above cells with fresh media one day post-transfection. Perform the intact-cell EFC assay two days after seeding the cells. To assay GLUT4-PL at the cell surface under basal growth conditions (serum-containing media, no exogenous insulin), proceed to step 3.

2) For insulin induction, add 20 μl/well of insulin diluted in media to 6× system concentration (e.g., add 20 μl of 6 μM insulin to the 100 μl liquid above cells to achieve an insulin system concentration of 1 μM). Return plate to incubator for 30 min. Starving cells of serum 2-to-4 hours prior to adding insulin diluted in serum-free media may achieve a larger insulin-response window.

3) Remove the media above the cells by aspiration. Add 50 μl/well thrombin solution (20 units/ml thrombin; 1×PBS; 0.1 mg/ml BSA; 10 mM each KF and NaAzide (NaN₃)). Return plate to incubator for 1 hr. Extending the incubation time up to a maximum of 1.5 hrs may increase signal.

4) Add 80 μl/well EA solution (prepared by mixing 1 part EA Reagent (DiscoveRx, Corp. Fremont, Calif.) with 3 parts 1×PBS; 1.83 mM MgSO₄; 10 mM each KF and NaAzide). Gently tap plate to mix reagents. Return plate to incubator for 1 hr.

5) Add 30 μl/well CL Substrate. Gently tap plate to mix reagents. Incubate at room temp protected from light. Readings are taken at periodic intervals from 15 min to 1 hr on a luminescence plate reader.

As evidenced by the above results and description, the subject methods provide simple assays employing conventional reagents and readers for determining the population of proteins on a surface. Where both the wild-type and fusion protein are being simultaneously expressed, one can provide a correlation between the value obtained with the fusion protein and the total cell membrane protein, if desired, using an immunoassay. Once the correlation has been established, one can rapidly determine the total population of the cell membrane protein by using the value obtained from the fusion protein and the graph as obtained with the values from the immunoassay.

The subject method provides a rapid and simple approach to determining cell membrane protein populations that can be used for single determinations or for high throughput screening. With the amplification obtained using an enzyme having a high turnover rate and measuring fluorescent or chemiluminescent products, accurate results with small differences can be readily determined.

All references referred to in the text are incorporated herein by reference as if fully set forth herein. The relevant portions associated with this document will be evident to those of skill in the art. Any discrepancies between this application and such reference will be resolved in favor of the view set forth in this application.

Although the invention has been described with reference to the above examples, it will be understood that modifications and variations are encompassed within the spirit and scope of the invention. Accordingly, the invention is limited only by the following claims.

1. A method for determining the population of a cell membrane protein bound to a cell membrane, employing a cell having a protein fusion construct comprising a signal producing peptide linked to at least an exofacial portion of said cell membrane protein through a protease recognition sequence, said signal producing peptide comprising an enzyme donor fragment capable of complexing with an enzyme acceptor fragment to form an active enzyme when not bound to said cell membrane or two binding sites for binding entities when brought together by binding to said signal producing peptide, said method comprising:
adding a protease that cleaves said protease recognition site to said cell, whereby said signal producing peptide is released from said cell membrane; and
assaying for said released signal producing peptide, wherein the signal produced with said signal producing peptide is related to the amount of said cell membrane protein population.

2. A method according to claim 1, wherein said cell is a mammalian cell.

3. A method according to claim 1, wherein said two binding entities are a pair of enzymes related by the product of one being the substrate of the other, a light absorbing and energy transfer entity and a energy accepting and light emitting entity, or a metastable species producing entity and an entity that reacts with said metastable species and produces light.

4. A method according to claim 1, wherein said signal producing peptide is an enzyme donor fragment.

5. A method according to claim 4, wherein said enzyme donor fragment is a β-galactosidase fragment.

6. A method according to claim 5, wherein said β-galactosidase fragment independently complexes with said enzyme acceptor fragment.

7. A method according to claim 1, wherein said protein fusion construct is expressed from an expression construct transiently or stably introduced into said cell.

8. A method of determining the effect of a change of environment on the population of a cell membrane protein bound to a cell membrane employing a cell having a protein fusion construct comprising a signal producing peptide linked to at least an exofacial portion of said cell membrane protein through a protease recognition sequence or sequences, said signal producing peptide comprising an enzyme donor fragment capable of complexing with an enzyme acceptor fragment to form an active enzyme when not bound to said cell membrane, said method comprising:
effecting said change of environment to said cell;
adding a protease to said cell whereby said signal producing peptide is released from said cell membrane;
assaying for said released signal producing peptide with said enzyme acceptor fragment and substrate, wherein the amount of product produced from said substrate is related to the amount of said cell membrane protein population; and
comparing the amount of product produced in the presence and absence of said change of environment.

9. A method according to claim 8, wherein said change of environment is the addition of a drug to said cell.

10. A method according to claim 8, wherein said signal producing peptide is a β-galactosidase fragment.

11. A method according to claim 10, wherein said β-galactosidase fragment independently complexes with said enzyme acceptor fragment.

12. A method according to claim 8, wherein said protein fusion construct is expressed from an expression construct transiently or stably introduced into said cell.

13. A nucleic acid comprising in the 5′-3′ direction a sequence encoding a cell membrane protein linked to an enzyme fragment through a protease recognition sequence and a signal leader sequence.

14. A nucleic acid according to claim 13, wherein said cell membrane protein comprises at least one sequence encoding a transmembrane amino acid sequence or an amino acid sequence that can be a substrate for membrane attachment via post-translational modification.

15. A protein encoded by a nucleic acid according to claim 13.

16. A cell comprising a nucleic acid according to claim 13.

17. A kit comprising a nucleic acid according to claim 13, an enzyme acceptor sequence, a protease that cleaves said protease recognition sequence and optionally a chemiluminescent or fluorescent substrate for the enzyme formed by the complexing of said enzyme fragment and said enzyme acceptor.

18. A kit according to claim 17, wherein said enzyme fragment and said enzyme acceptor complex to form β-galactosidase.
